(19) United States
(12) Patent Application Publication
(10) Pub. No.: US 2020/0222116 A1
(43) Pub. Date: Jul. 16, 2020

(54) SYSTEMS AND METHODS FOR LASER-ASSISTED PLACEMENT OF ORTHOPEDIC IMPLANTS

(71) Applicant: MiRus LLC, Marietta, GA (US)

(72) Inventor: Jay Yadav, Sandy Springs, GA (US)

(21) Appl. No.: 16/741,564

(22) Filed: Jan. 13, 2020

Related U.S. Application Data
(60) Provisional application No. 62/791,492, filed on Jan. 11, 2019.

(51) Int. Cl. A61B 18/20; A61B 34/30; A61B 34/00; A61B 18/22; A61B 17/16; A61B 17/34; A61B 17/88; A61B 17/86; A61B 34/20

(52) U.S. Cl. CPC A61B 18/201; A61B 34/30; A61B 34/25; A61B 18/22; A61B 17/1637; A61B 2017/00973; A61B 17/8875; A61B 17/864; A61B 34/20; A61B 18/203; A61B 17/3472

(57) ABSTRACT

Devices, systems, and methods for placing orthopedic implants are disclosed. In one aspect, a tool for laser assisted placement of an orthopedic implant includes a cannulated body having a proximal end and a distal end. The cannulated body includes an inner channel extending between the proximal and distal ends. The tool also includes an optical cable arranged within the inner channel of the cannulated body, and a tip arranged at the distal end of the cannulated body. The tip is configured to allow passage of laser radiation through the distal end of the cannulated body.

### CROSS REFERENCE TO RELATED APPLICATIONS

This application claims the benefit of U.S. provisional patent application No. 62/791,492, filed on Jan. 11, 2019, and entitled “SYSTEMS AND METHODS FOR LASER-ASSISTED PLACEMENT OF ORTHOPEDIC IMPLANTS,” the disclosure of which is expressly incorporated herein by reference in its entirety.

### TECHNICAL FIELD

The present disclosure relates generally to orthopedic surgery and, more particularly, to a system and method for laser-assisted placement of orthopedic implants.

### BACKGROUND

Many orthopedic surgeries, such as those involving the spine, are complex procedures that require a high degree of precision. For example, the spine is in close proximity to delicate anatomical structures such as the spinal cord and nerve roots. Pedicle screw placement is among the most effective schemes for stabilizing the spine. With pedicle diameters ranging from 4 to 20 mm, screw fixation into the pedicle requires great precision to avoid skiving, cortex violation and damage to surrounding nerves and/or spinal cord. Compounding the problem is limited surgical exposure and visibility, particularly in the case of minimally invasive procedures. Consequently, the risk of misplaced implants or other complications is high.

Current means of implant placement relies on preparation (cannulation) of the implant site using rudimentary mechanical instrumentation such as needles, drills, and/or burrs and is highly dependent on the skill/experience level of the surgeon. The reaction forces such mechanical instrumentation encounter when in contact with bony surfaces can cause skiving and deviations from the desired trajectory. Rotary drills and burrs can tend to wander off trajectory and require anti-skiving and soft-tissue protection strategies such as use of drill guides and/or sleeves. For the above reasons current methods of implant placement lack consistency and precision and/or require time consuming preparatory steps. Such uncertainty in implant placement has a negative impact on long term clinical outcomes, patient quality of life, and the ability to predict and control costs associated with surgery, recovery, and rehabilitation.

To overcome the above issues associated with mechanical methods for placing implants, use of alternate high precision technologies such as lasers that improve placement accuracy and are compatible with existing standard procedures would be advantageous. The presently disclosed systems and associated methods for laser-assisted placement of orthopedic implants are directed at overcoming one or more of the problems set forth above and/or other problems in the art.

### SUMMARY

According to one aspect, the present disclosure is directed to a method for laser-assisted placement of implants. The method includes delivering laser radiation via an optical cable or fiber passed through a cannulated instrument and/or screw to a region of the bone that is targeted for removal and/or is being prepared for implant placement. In an alternate embodiment the laser radiation is delivered through air without an optical cable or fiber by aligning the laser to the cannula of the instrument. The instrument can either be aligned in the direction of the target region on the bone and/or be in close proximity or in contact with it. The method further includes selection of an appropriate laser wavelength and controlling the laser power, pulse duration, pulse frequency, and/or beam diameter such that removal rate and area of bone removed is tailored to the specific goals of the procedure. The method further includes switching between or combining mechanical and laser modes of bone removal so as to achieve optimal placement of the implant.

In accordance with another aspect, the present disclosure is directed to an instrument for laser-assisted placement of an implant. In one embodiment, the instrument includes a cannula through which an optical cable or fiber capable of transmitting laser radiation is passed. The optical cable is used to deliver laser radiation to the bone. In another embodiment the laser radiation is delivered through air through the cannula without an optical cable. In some embodiments the tool is a cannulated cylindrical tube. In other embodiments the instrument is one of standard cannulated surgical instruments such as a Jamshidi needle, awl, probe or tap. This allows the user to use the mechanical abilities of the tool along with laser radiation to accomplish the surgical goals. In yet another embodiment the tool is a cannulated screw driver configured to couple to a cannulated screw. The laser radiation is passed through both the screw driver and screw in this case. This also allows for laser radiation to be utilized along with the normal functionality of a traditional surgical screw driver.

In accordance with another aspect, the present disclosure is directed to a system for laser assisted placement of an orthopedic implant. The system includes a cannulated instrument and a laser sub-system including laser source, electronics, mechanical components, cooling sub-systems for both the laser source and irradiated bone, optics, and in certain embodiments articulating arms. The cannulated surgical instrument through which the laser radiation is passed and allows utilization of laser radiation in conjunction with mechanical means to place the implant into the bony anatomy. The laser sub-system generates, transmits, and focuses laser radiation of a selected wavelength in the desired direction and/or location after passage through the cannula of the instrument. The passage of laser radiation through the cannula can be through air or via optical cable or fiber that is coupled to the laser sub-system and passed through the cannula. The laser sub-system is equipped with a means to control the laser power, beam diameter, duration, pulse rate, and/or duty cycle. The laser sub-system further has means for a user to interact with it for the purpose on controlling the laser such as buttons, foot pedals, input/output (I/O) devices, and/or a user interface on a computer monitor. The system can also include tubular retractors or tissue protectors for minimally invasive procedures. The system can also include a navigation system and/or robotically controlled arm and/or guide for guidance and/or precise positioning of the instrument.

An example tool for laser-assisted placement of an orthopedic implant is described herein. The tool includes a cannulated body having a proximal end and a distal end, where the cannulated body has an inner channel extending between the proximal and distal ends. The tool also includes an optical cable arranged within the inner channel of the cannulated body, and a tip arranged at the distal end of the cannulated body, where the tip is configured to allow passage of laser radiation through the distal end of the cannulated body.

An example system for laser-assisted placement of an orthopedic implant is also described herein. The system includes a laser sub-system comprising a laser source, and a cannulated tool having a proximal end and a distal end. The cannulated tool has an inner channel extending between the proximal and distal ends. The laser source is configured to deliver laser radiation through the inner channel of the cannulated tool. In some implementations, the system optionally further includes an optical cable arranged within the inner channel of the cannulated body, where the optical cable is coupled to the laser source and configured to deliver the laser radiation through the inner channel of the cannulated tool.

In some implementations, the system optionally further includes a surgical robot including a robotic arm, where the cannulated tool is attached to the robotic arm. Optionally, the system further includes a guide attached to the robotic arm, where the cannulated tool is configured to slide in the guide.

In some implementations, the system optionally further includes a navigation system configured to guide the cannulated tool during a surgical procedure.

An example method for laser-assisted placement of an orthopedic implant is also described herein. The method includes providing a cannulated tool, aligning the cannulated tool with a target location on a bone, and delivering laser radiation through the cannulated tool to the target location on the bone, where the laser radiation is configured to cause removal of the bone in proximity to the target location.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 provides a diagrammatic view of an exemplary system for laser-assisted placement of an orthopedic implant consistent with certain disclosed embodiments.

FIG. 2 provides a diagrammatic view of an alternate exemplary system for laser-assisted placement of an orthopedic implant consistent with certain disclosed embodiments.

FIG. 3 provides a diagrammatic view of an alternate exemplary system for laser-assisted placement of an orthopedic implant consistent with certain disclosed embodiments.

FIG. 4 provides a diagrammatic view of another alternate exemplary system for laser-assisted placement of an orthopedic implant consistent with certain disclosed embodiments.

FIG. 5 provides a diagrammatic view of another alternate exemplary system for laser-assisted placement of an orthopedic implant consistent with certain disclosed embodiments.

FIG. 6 provides a diagrammatic view of another alternate exemplary system for laser-assisted placement of an orthopedic implant consistent with certain disclosed embodiments.

### DETAILED DESCRIPTION

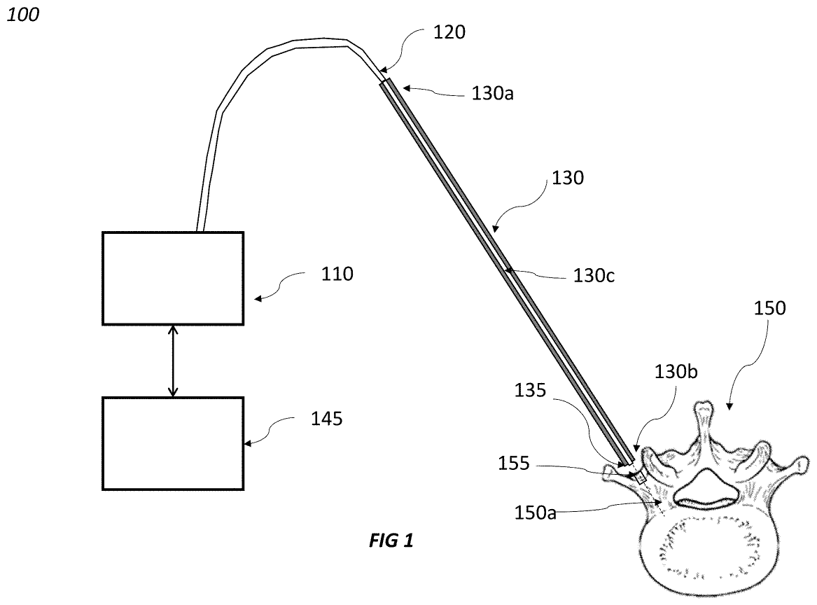

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Throughout the present disclosure, the terms “bone” and “vertebrae” may be used interchangeably. The bone depicted in FIGS. 1-6 is a vertebrae, but the present disclosure contemplates applying the disclosed tool, system, and methods to bones other than vertebrae. Additionally, throughout the present disclosure the “tool” may also be referred to as “instrument” and “tool” may be referred to interchangeably with “cannulated tool.”

Different configurations of tool are contemplated. In some implementations, the tool is a surgical implement. A surgical screwdriver is a non-limiting example of a surgical implement. According to some implementations, the tool can be a surgical screwdriver that is configured to couple to a cannulated screw. Other tools and surgical instruments are contemplated, including Jamshidi needles, awls, probes and taps.

FIG. 1 provides a diagrammatic view of exemplary system 100 for laser-assisted placement of an orthopedic implant. The system includes a laser sub-system 110 coupled to an optical cable 120. Optical cable 120 can be a suitable flexible or semi-rigid fiber or hollow tube designed to facilitate transmission of laser radiation. The system also includes a cannulated tool 130 having a proximal end 130a and a distal end 130b. An inner channel 130c extends between the proximal end 130a and the distal end 130b. This disclosure contemplates that an optical cable 120 can be arranged within the inner channel 130c of the cannulated tool 130 as shown in FIG. 1, where the optical cable 120 is coupled to the laser source and configured to deliver the laser radiation through the inner channel 130c of the cannulated tool 130. The laser radiation exits the cannulated tool 130 via a tip 135 arranged at the distal end 130b of the cannulated tool 130. According to some implementations, the tip 135 is configured to retain the optical cable 120 within the inner channel 130c of the cannulated tool 130. For example, a lip can be provided around the tip 135. The lip creates an opening for the laser radiation to exit that is smaller than the outer diameter of the optical cable 120. The optical cable 120 is therefore retained within the inner channel 130c by the lip. It should be understood that the lip is provided only as an example means for retaining the optical cable 120 within the inner channel 130c.

Optionally, a reflective material can be arranged on a portion of the inner channel 130c of the cannulated tool 130 in some implementations. For example, the reflective material can be provided on the surface (or portions thereof) of the inner channel 130c. This disclosure contemplates that the reflective material can be any material capable of reflecting the laser radiation. In an exemplary embodiment the optical cable 120 is a hollow glass tube whose internal surfaces are coated with a reflective material. For example, in the case where the laser is CO₂ laser a coating of silver (Ag) may be utilized. It should be understood that Ag is only provided as an example reflective material. This disclosure contemplates using other reflective materials with the systems and methods described herein, for example, a reflective material capable of the laser radiation produced by the laser source. In another embodiment the optical cable 120 is a fiber optic cable made of a material such as sapphire or silica. Non-limiting examples of optical cable 120 diameters include the range from about 0.2 to 5 mm.

The laser sub-system 110 can include any laser source suitable for ablation of bone. Different wavelengths of laser are contemplated by the present disclosure, including ultra-violet laser sources and infra-red laser sources. Example wavelengths suitable for bone ablation are infra-red wavelengths such as those produced by excitation of CO₂, Er:YAG, Ho:YAG, and Nd:YAG. In some cases ultra-violets wavelengths such as those produced by excitation of XeCl can be utilized. The laser sub-system 110 can include appropriate solid-state or gas-based laser sources with associated control circuitry. It should be understood that materials excited to produce the infra-red and ultra-violet wavelengths above are only provided as non-limiting examples. Some embodiments described herein include a user interface 145 configured to receive commands from the users. The user interface 145 can be coupled to the laser sub-system 110. The laser sub-system 110 and user interface 145 discussed above can be coupled through one or more communication links. This disclosure contemplates the communication links are any suitable communication link. For example, a communication link can be implemented by any medium that facilitates data exchange between the laser sub-system 110 and user interface 145 including, but not limited to, wired, wireless and optical links. Additionally, the user interface 145 can include an input device such as a touch screen and/or foot pedal.

This disclosure contemplates that the laser sub-system 110 can be further configured to control at least one of power, pulse duration, pulse frequency, or beam width of the laser radiation. This disclosure contemplates that a user can use the laser sub-system 110 and tool 130 to deliver laser radiation of sufficient power, characteristics, and duration necessary for a particular surgical procedure, for example, to remove bone in and around the location 155 of laser radiation delivery, e.g., in proximity to the pedicle 150a in FIG. 1. In an exemplary embodiment, the laser sub-system 110 is capable of varying the power over a range of about 20-200 W. Alternatively or additionally, the laser sub-system 110 can be capable of delivering the energy in pulses of duration ranging from 100 millisecond (ms) to 1 microsecond (μs) with repetition rates from 1 Hertz (Hz) to 1000 Hz. This disclosure contemplates that pulsing techniques enabling nano, pico, and femtosecond pulsing can also optionally be used with the systems and methods described herein. In general shorter pulses will results smaller amounts of bone charring, which may be desirable in some implementations. Alternatively or additionally, the diameter of the beam produced by the laser sub-system 110 can also be variable. For example, the beam diameter can include the range from about 0.1 millimeter (mm) to about 5 mm. A person of skill in the art would understand how to control the laser sub-system 110 to select characteristics of the laser radiation to achieve the desired results (e.g., bone removal).

The laser sub-system 110 can optionally include a cooling system for cooling the region of laser ablation on the bone. For example, the laser sub-system 110 can be equipped with compressed air or other suitable gas that is passed through the cannula of tool 130 or through optical cable 120 or directly delivered to the ablated site.

In another embodiment sterile fluid such as saline instead of gas can be utilized. An exemplary laser sub-system 110 that can used in system 100 is the Ultra MD Series CO₂ Laser by Laser Engineering, Nashville Tenn. It should be understood that other laser sub-systems can be used with the tool, systems, and methods described herein. Optical cable 120 is passed through tool 130 and delivers laser radiation to the desired target location 155 on bone 150. The laser radiation is turned on for a sufficient duration to ablate the target location 155 to a depth necessary to facilitate placement of an orthopedic implant (not shown) into pedicle 150a. Typical radiation times can range from about 3 to 30 seconds for ablations depths of about 3-30 mm. For example, the system can be used to breach or indent the cortex of vertebra 150 to open up an entry point for a pedicle screw. The tool 130 can be a generic tubular instrument of inner diameter in the range of about 0.5 to 2 mm and outer diameter in the range of about 1-5 mm. Alternatively, tool 130 can be a customized instrument with sufficient cannular diameter for passage of laser radiation. The inner surfaces of tool 130 can optionally be coated with reflective material to aid in the transmission of laser radiation.

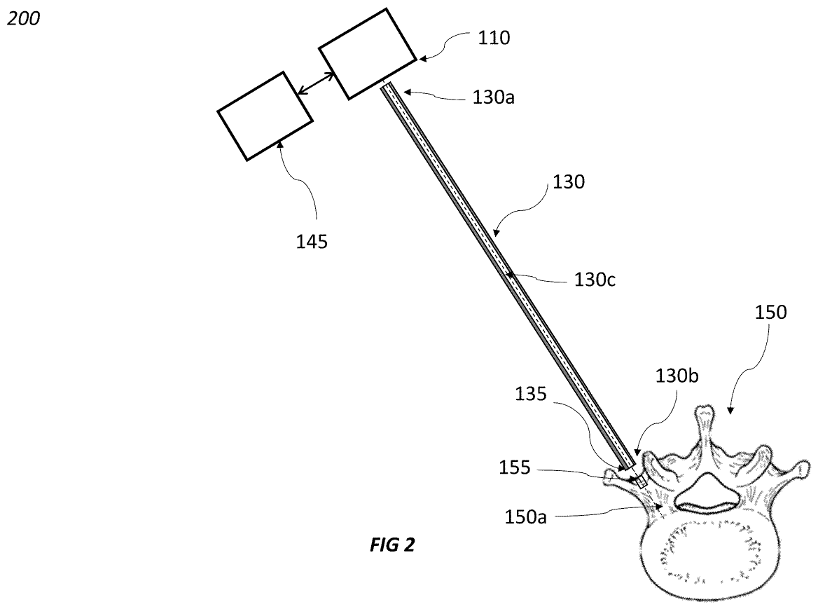

FIG. 2 provides a diagrammatic view of another exemplary system 200 for laser assisted placement of an orthopedic implant. The system includes a laser sub-system 110 including a laser source and a cannulated tool 130. In this embodiment the laser beam emanating from laser-subsystem 110 is aligned to pass through tool 130 (e.g., the laser radiation passes through air). As described above, the cannulated tool 130 has a proximal end 130a and a distal end 130b. An inner channel 130c extends between the proximal end 130a and the distal end 130b. The laser radiation exits the cannulated tool 130 via a tip 135 arranged at the distal end 130b of the cannulated tool 130. For example, laser sub-system 110 can be equipped with an articulated arm for delivery of the laser beam which may then be mechanically aligned and coupled with tool 130. Additionally, a user interface 145 can be configured to receive commands from the users, and the user interface 145 can be coupled to the laser sub-system 110 as described herein.

Alternatively, sub-system 110 can include a pointing laser such as a low power HeNe for targeting and alignment. This disclosure contemplates that the laser sub-system 110 can be further configured to control at least one of power, pulse duration, pulse frequency, or beam width of the laser radiation as described herein. The tool 130 can be used to remove bone in and around the location 155 of laser radiation delivery, e.g., in proximity to the pedicle 150a in FIG. 2. Further, different wavelengths of laser are contemplated by the present disclosure, including ultraviolet laser sources and infrared laser sources. A reflective material can optionally be arranged on a portion of the inner channel 130c of the cannulated tool 130.

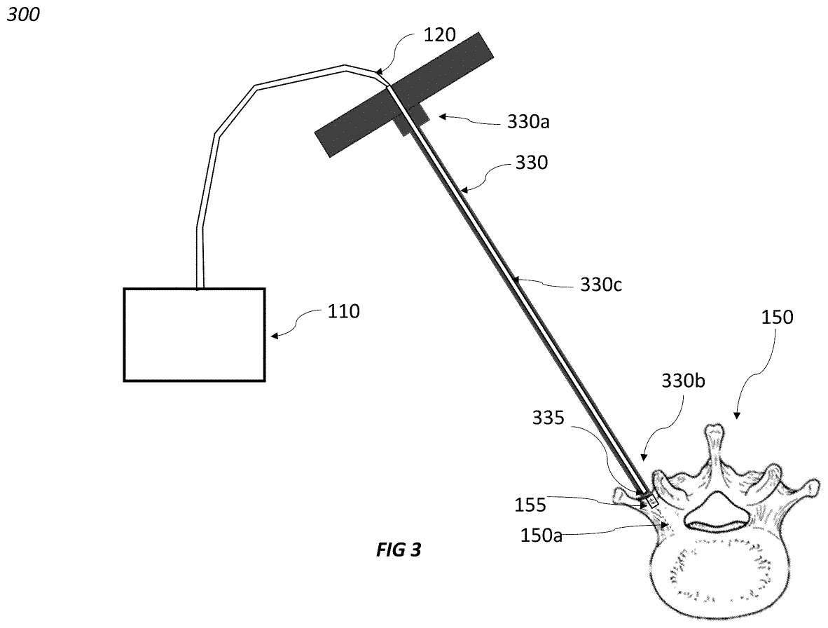

FIG. 3 provides a diagrammatic view of another exemplary system 300 for laser assisted placement of an orthopedic implant. The system includes of a laser sub-system 110 coupled to an optical cable 120 and a cannulated surgical instrument 330 such as a Jamshidi needle. As described above, the cannulated instrument 330 has a proximal end 330a and a distal end 330b. An inner channel 330c extends between the proximal end 330a and the distal end 330. The optical cable 120 is arranged within the inner channel 330c of the cannulated instrument 330 as shown in FIG. 3, where the optical cable 120 is coupled to the laser source and configured to deliver the laser radiation through the inner channel 330c of the cannulated instrument 330. The laser radiation exits the cannulated instrument 330 via a tip 335 arranged at the distal end 330b of the cannulated instrument 330. The cable 120 is passed through a cannulated instrument 330 and delivers laser radiation to the bone 150 at target location 155. This disclosure contemplates that the laser sub-system can be further configured to control at least one of power, pulse duration, pulse frequency, or beam width of the laser radiation as described herein.

The normal function of surgical instrument 330 can be combined with laser radiation can be utilized to remove bone at a desired location and/or depth. For example, as shown in FIG. 3, the surgical instrument 330 can use laser radiation in conjunction with mechanical interaction to breach and cannulate the cortex of vertebra 150 to facilitate placement of a pedicle screw into pedicle 150a.

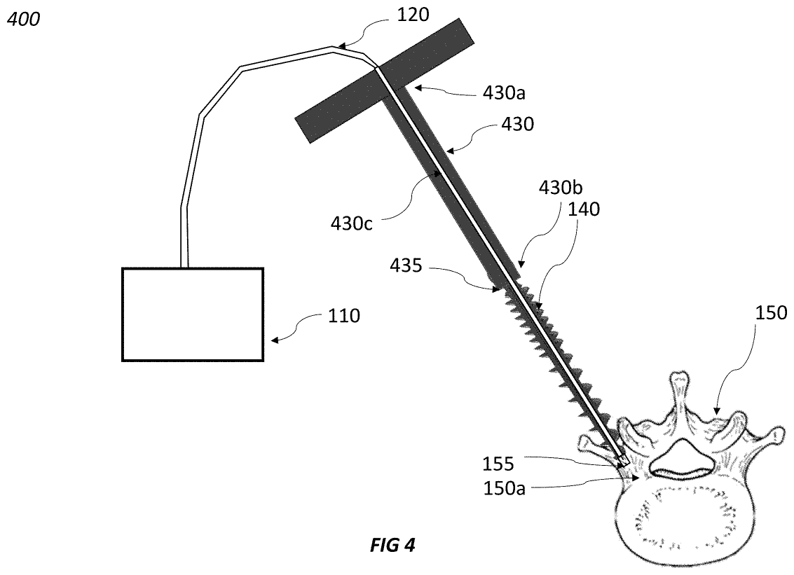

FIG. 4 provides a diagrammatic view of another alternate exemplary system 400 for laser-assisted placement of an orthopedic implant consistent with certain disclosed embodiments. The system includes a laser sub-system 110 coupled to optical cable 120. The optical cable 120 is passed through cannulated screw driver 430 and screw 140, which is also cannulated, and delivers laser radiation to the bone 150 at the target location 155. As described above, the cannulated screw driver 430 has a proximal end 430a and a distal end 430b. An inner channel 430c extends between the proximal end 430a and the distal end 430b. The distal end 430b of the screw driver 430 is coupled to the cannulated screw 140. The optical cable 120 can be arranged within the inner channel 430c of the cannulated screw driver 430 and also arranged within the cannula of the screw 140 as shown in FIG. 4. The optical cable 120 is coupled to the laser source and configured to deliver the laser radiation through the inner channel 430c of the cannulated screw driver 430 and cannulated screw 140. The laser radiation exits the cannulated screw driver 430 and screw 140 at a distal end thereof. The normal function of the screw driver 430 and screw 140 combined with laser radiation can be utilized to remove bone at a desired location and/or depth. For example, the screw driver 430 can be used to drive a pedicle screw 140 into pedicle 150a of vertebra 150 using a combination of mechanical interaction and laser radiation. It be understood that the screw driver 430 can either be conventional manual driver or a power driver.

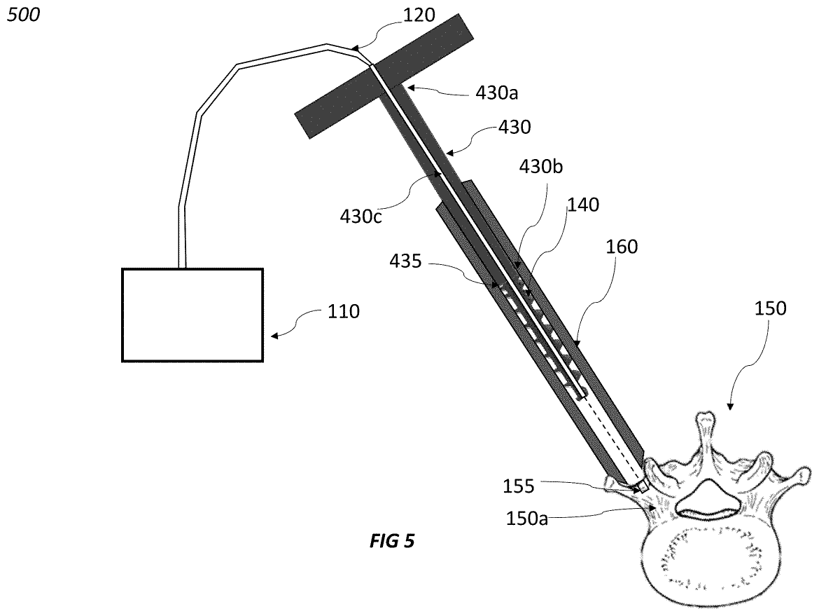

FIG. 5 provides a diagrammatic view of another alternate exemplary system 500 for laser-assisted placement of an orthopedic implant consistent with certain disclosed embodiments. As described above, the cannulated screw driver 430 has a proximal end 430a and a distal end 430b. An inner channel 430c extends between the proximal end 430a and the distal end 430b. The distal end 430b of the screw driver 430 is coupled to the cannulated screw 140. The optical cable 120 can be arranged within the inner channel 430c of the cannulated screw driver 430 and also arranged within the cannula of the screw 140 as shown in FIG. 5. The optical cable 120 is coupled to the laser source and configured to deliver the laser radiation through the inner channel 430c of the cannulated screw driver 430 and cannulated screw 140. The laser radiation exits the cannulated screw driver 430 and screw 140 at a distal end thereof. A guide 160 is also included in the system of FIG. 5. The guide 160 facilitates alignment of screw driver 430 to the desired trajectory and can also be a tissue protector or tubular retractor for minimally invasive surgery. The guide 160 can also be configured to facilitate access to the bone through soft tissue. The guide 160 can be configured to be manipulated by a robotic arm (not shown in FIG. 5), and the robotic arm can be part of a surgical robot (not shown in FIG. 5). Similar to the system of FIG. 4, the normal function of the screw driver 430 and screw 140 combined with laser radiation can be utilized to remove bone at a desired location and/or depth. For example, the screw driver 430 can be used to drive a pedicle screw 140 into pedicle 150a of vertebra 150 using a combination of mechanical interaction and laser radiation. It should be understood that screw driver 430 is provided only as an example tool or instrument in FIG. 5. This disclosure contemplates that other tools or instrument, including but not limited to those shown in FIGS. 1-4, can be used with the system 500 of FIG. 5.

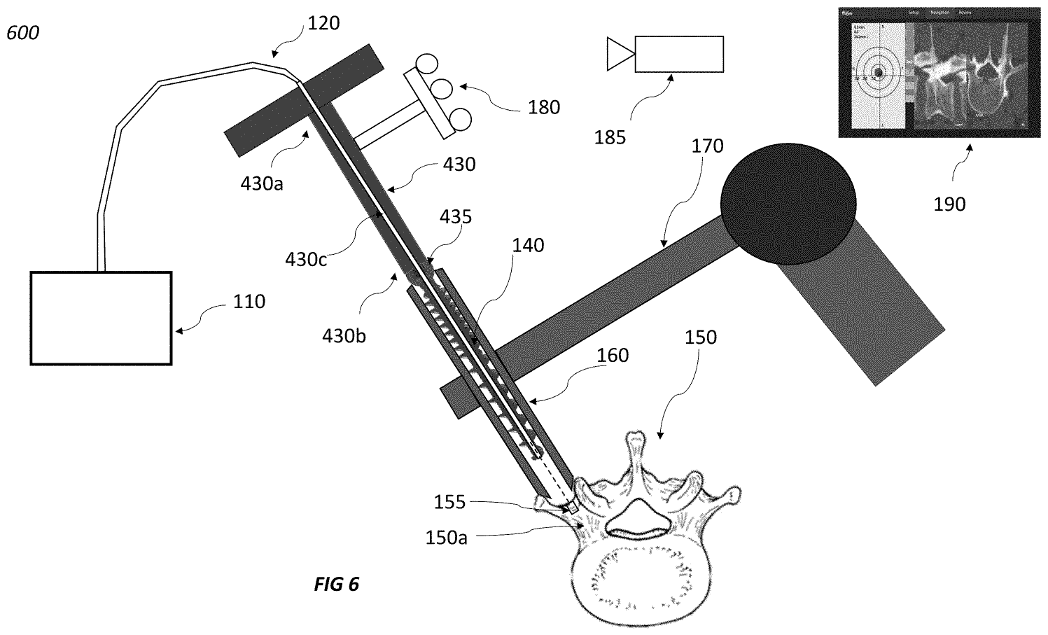

FIG. 6 provides a diagrammatic view of another alternate exemplary system 600 for laser-assisted placement of an orthopedic implant consistent with certain disclosed embodiments. In this embodiment the system comprises navigation and/or robotic sub-systems for computer assisted surgery. Surgical navigation systems are known in the art and are therefore not described in detail herein. Additionally, robotic surgical systems are known in the art and are therefore not described in detail herein. As described above, the cannulated screw driver 430 has a proximal end 430a and a distal end 430b. It should be understood that screw driver 430 is provided only as an example tool or instrument in FIG. 6. This disclosure contemplates that other tools or instrument, including but not limited to those shown in FIGS. 1-4, can be used with the system 600 of FIG. 6. An inner channel 430c extends between the proximal end 430a and the distal end 430b. The distal end 430b of the screw driver 430 is coupled to the cannulated screw 140. The optical cable 120 can be arranged within the inner channel 430c of the cannulated screw driver 430 and also arranged within the cannula of the screw 140 as shown in FIG. 6. The optical cable 120 is coupled to the laser source and configured to deliver the laser radiation through the inner channel 430c of the cannulated screw driver 430 and cannulated screw 140. The laser radiation exits the cannulated tool 130 and screw 140 at a distal end thereof. Optionally, the system can include a guide 160 can be slidably coupled to screw driver 430. The guide 160 facilitates alignment and positioning of the screw driver 430. The guide 160 may also be used for tissue retraction/protection (e.g., a tissue protector or tubular retractor). This disclosure contemplates attaching the guide 160 to a robotic arm 170 of a robotic surgical system. FIG. 6 illustrates an implementation where the guide 160 is the end effector of the robotic system. It should be understood that the tool 130 itself can be directly attached to the robotic arm 170 in other implementations.

The robotic sub-system can include a computer-controlled robotic system and a robotic arm 170. Any surgical robotic system that is designed to position an end effector relative to the patient's anatomy can be utilized in system 600. In an exemplary embodiment the robotic arm 170 is used to position the precisely position guide 160 which then facilitates proper placement of screw driver 430 with or without screw 140. Screw driver 430 can also be directly attached to the robot end effector in which case the robot motion is controlled along the desired trajectory and/or to the desired position. A non-limiting example of an attachment technique is a quick connect mechanism. Alternatively, the screw driver 430 slides through a guide 160 as shown in FIG. 6 that acts as the robot end effector. Typical steps prior to use of such robotic positioning can be utilized including image registration, instrument calibration, robot calibration, instrument calibration, and planning. The robotic arm 170 can be used in conjunction with any of the systems described above (FIG. 1-5). For example, in the system of any one of FIGS. 1-5, the robotic arm 170 can be used to position tool (e.g., needle, screw driver, etc.). A non-limiting example of a robotic sub-system that can be used in the embodiment as described above is the Mazor-X robotic system by Medtronic, Dublin, Ireland. It should be understood that other robotic surgical systems can be used with the tool, systems, and methods described herein.

In another exemplary embodiment as shown in FIG. 6, system 600 includes a navigation sub-system in addition to components of the systems shown in FIGS. 1-5. The navigation sub-systems can include a camera 185 that can track the 3D position and orientation of screw driver 430 via fiducial 180 rigidly attached to it. The position of screw driver 430 relative to the anatomy is displayed on monitor 190. This facilitates precise positioning of screw driver 430. The Navigation sub-system can be utilized with or without the robotic sub-system described above. A non-limiting example navigation sub-system suitable for use with this embodiment is the StealthStation System by Medtronic, Dublin, Ireland. It should be understood that other navigation systems can be used with the tool, systems, and methods described herein. Steps prior to use of such navigation systems may include image registration, instrument calibration, robot calibration, instrument calibration, and planning.

With reference to FIGS. 1-6, the present disclosure contemplates methods for laser-assisted placement of an orthopedic implant, including providing a cannulated tool 130 (or surgical instrument 330 or screw driver 430), aligning the cannulated tool or instrument with a target location 155 on the bone 150, and delivering laser radiation through the cannulated tool or instrument to the target location 155 on the bone 150. The laser radiation is configured to cause removal of the bone in proximity to the target location 155 (e.g., in and around the region where laser radiation is delivered). In some implementations, at least a portion of the bone 150 is removed using the laser radiation. Some methods contemplated by the present disclosure include removing the at least a portion of the bone 150 using the cannulated tool or instrument. Additionally, implementations of the present disclosure include switching between mechanical and laser modes of bone removal. In some implementations of the present disclosure, mechanical and laser methods of bone removal can be combined. According to some implementations of the present disclosure, the portion of the bone 150 is removed concurrent with placement of the orthopedic implant. The present disclosure also contemplates aligning the cannulated tool or instrument in contact with the target location 155 on the bone 150, and/or the cannulated tool or instrument being aligned in proximity to the target location 155 on the bone 150.

According to some implementations of the present disclosure, the laser radiation is delivered through the cannulated tool or instrument using an optical cable 120 arranged within an inner channel of the cannulated tool or instrument. Additionally, this disclosure contemplates delivering laser radiation through the cannulated tool or instrument by aligning a laser source to an inner channel of the cannulated tool or instrument (e.g., through air without use of optical cable). The laser source can be part of a laser sub-system 110. The present disclosure also contemplates controlling at least one of a wavelength, power, pulse duration, pulse frequency, or beam width of the laser radiation based on the surgical procedure. Use of different wavelengths of laser radiation are contemplated by the present disclosure, including ultraviolet laser radiation and infrared laser radiation. According to some implementations of the present disclosure, the cannulated tool is a surgical instrument. Different surgical instruments are contemplated, including Jamshidi needles, awls, probes, and taps. The surgical instrument may also include a surgical screw driver configured to couple to a cannulated screw. The use of surgical robots to perform implementations of the method is also contemplated by the present disclosure. The surgical robot may include a robotic arm 170. For example, the cannulated tool or instrument can be controlled with the surgical robot. According to some implementations of the present disclosure, the cannulated tool or instrument can be guided during the procedure using a surgical navigation system. The surgical navigation system may include a camera 185 and/or a fiducial 180 that are used to position the tool or instrument.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A tool for laser-assisted placement of an orthopedic implant, comprising:
a cannulated body having a proximal end and a distal end, wherein the cannulated body comprises an inner channel extending between the proximal and distal ends;
an optical cable arranged within the inner channel of the cannulated body; and
a tip arranged at the distal end of the cannulated body, wherein the tip is configured to allow passage of laser radiation through the distal end of the cannulated body.

2. The tool of claim 1, wherein the tip is further configured to retain the optical cable within the inner channel of the cannulated body.

3. The tool of claim 1, further comprising a reflective material arranged on at least a portion of a surface of the inner channel of the cannulated body.

4. The tool of claim 1, wherein the tool is a surgical instrument.

5. The tool of claim 4, wherein the surgical instrument is a Jamshidi needle, an awl, a probe, or a tap.

6. The tool of claim 4, wherein the surgical instrument is a screw driver configured to couple to a cannulated screw.

7. A system for laser-assisted placement of an orthopedic implant, comprising:
a laser sub-system comprising a laser source; and
a cannulated tool having a proximal end and a distal end, wherein the cannulated tool comprises an inner channel extending between the proximal and distal ends, wherein the laser source is configured to deliver laser radiation through the inner channel of the cannulated tool.

8. The system of claim 7, further comprising an optical cable arranged within the inner channel of the cannulated body, wherein the optical cable is coupled to the laser source and configured to deliver the laser radiation through the inner channel of the cannulated tool.

9. The system of claim 7, wherein the laser source is aligned with the cannulated tool to pass the laser radiation through the inner channel of the cannulated tool.

10. The system of claim 7, wherein the laser sub-system is further configured to control at least one of power, pulse duration, pulse frequency, or beam width of the laser radiation.

11. The system of claim 7, further comprising a user interface operably coupled to the laser sub-system, wherein the user interface is configured to receive commands from a user.

12. The system of claim 7, wherein the laser source is an ultraviolet laser source.

13. The system of claim 7, wherein the laser source is an infrared laser source.

14. The system of claim 7, wherein the cannulated tool is a surgical instrument.

15. The system of claim 14, wherein the surgical instrument is a Jamshidi needle, an awl, a probe, or a tap.

16. The system of claim 14, wherein the surgical instrument is a screw driver configured to couple to a cannulated screw.

17. The system of claim 7, further comprising a surgical robot comprising a robotic arm, wherein the cannulated tool is attached to the robotic arm.

18. The system of claim 7, further comprising a surgical robot comprising a guide and a robotic arm, wherein the guide is attached to the robotic arm, and wherein the cannulated tool is configured to slide in the guide.

19. The system of claim 7, further comprising a navigation system configured to guide the cannulated tool during a surgical procedure.

20. A method for laser-assisted placement of an orthopedic implant, comprising:
providing a cannulated tool;
aligning the cannulated tool with a target location on a bone; and
delivering laser radiation through the cannulated tool to the target location on the bone, wherein the laser radiation is configured to cause removal of the bone in proximity to the target location.

21-36. (canceled)
